(19) United States
(12) Patent Application Publication
(10) Pub. No.: US 2004/0265264 A1
(43) Pub. Date: Dec. 30, 2004

(54) Olive oil PEG-7 carboxylates in cosmetic cleansing recipes

(75) Inventors: Martin Sugar, Hamburg (DE); Andreas Clausen, Hamburg (DE); Stephan Ruppert, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/786,636

(22) Filed: Feb. 25, 2004

Related U.S. Application Data
(63) Continuation of application No. PCT/EP02/09308, filed on Aug. 21, 2002.

(30) Foreign Application Priority Data
Aug. 25, 2001 (DE) 101 41 781.0

(51) Int. Cl. A61K 7/075; A61K 7/08

(52) U.S. Cl. 424/70.22; 424/70.24

(57) ABSTRACT

Wash-active cosmetic or dermatological formulations comprising sodium PEG-7 olive oil carboxylate with an active content of 0.01 to 1.8% by weight, based on the total weight of the formulation, in addition to other auxiliaries, active compounds and additives, provide cosmetic cleansing compositions having desirable properties.

### CROSS-REFERENCE TO RELATED APPLICATION

This is a continuation application of PCT/EP02/09308, filed Aug. 21, 2002, which is incorporated herein by reference in its entirety, and also claims the benefit of German Priority Application No. 101 41 781.0, filed Aug. 25, 2001.

### FIELD OF THE INVENTION

The present invention relates to the use of olive oil PEG-7 carboxylic acids and their salts as mild anionic surfactants in cosmetic cleansing compositions.

### BACKGROUND OF THE INVENTION

Surfactants are of great importance as wash-active substances in cosmetic cleansing compositions. As a result of their specific molecular structure with in each case a hydrophilic (water-attracting) and hydrophobic (water-repellent) grouping in the same molecule, they ensure reduction in the surface tension of the water, wetting of the skin, facilitation of the removal of dirt and foam regulation.

A distinction is made between four classes of surfactants:

The nonionic surfactants form a first class. These include fatty alcohol ethoxylates [RO(CH₂CH₂O)ₙH], fatty acid monoethanolamides [RCONHCH₂CH₂OH] and alkyl polyglycosides (APGs)

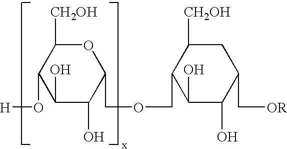

R=fatty acid radical

The amphoteric surfactants form a second class. These are compounds which contain both a cationic function, usually a quaternary nitrogen, and an anionic function, usually a carboxylate group. Their charge state depends on the pH. They include alkylaminobetaines

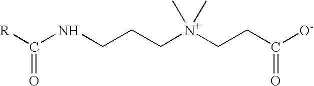

and alkylamidoglycinates.

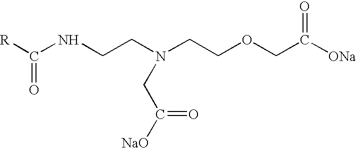

R=fatty acid radical

The group of cationic surfactants comprises compounds which contain at least one quaternary nitrogen atom. These include, for example, alkylamines, alkylimidazoles, and ethoxylated amines.

The group of anionic surfactants is formed from sulfates, sulfonates and carboxylates, i.e. salts of esters of sulfuric acid, and salts of sulfonic and carboxylic acids.

The most common surfactants of this class are derived from lauryl alcohol (C₁₂H₂₅OH). These are the sodium salt of lauryl sulfate (sodium lauryl sulfate, SLS) with the structure C₁₂H₂₅OSO₃ and ethylene glycol ethers of the alcohol, which are also esterified with sulfuric acid (sodium laureth sulfate, SLES). These have the structure:

C₁₂H₂₅(OCH₂CH₂)ₙOSO₃.

However, commercially available sodium lauryl ether-sulfate (sodium polyoxyethylene lauryl sulfate), which was used in the present invention, is as a rule a mixture of substances, the structures of which obey the general formula

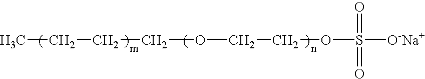

wherein m can assume the numbers 4 to 6 and n can assume the numbers 0 to 10.

These compounds have excellent wash-active properties and a good foam formation capacity. However, they are irritating to the skin and mucous membranes in higher doses. Furthermore, they lead to a reduction in skin moisture and to an increase in the transepidermal water loss (TEWL). Because of the good availability, the excellent washing properties and last but not least because of the acceptable price, it is scarcely possible to dispense with this surfactant completely.

### SUMMARY OF THE INVENTION

The object of the present invention was therefore to increase the skin tolerability of wash-active cosmetic and/or dermatological formulations. In particular, the skin tolerability of formulations which comprise sodium laureth ether-sulfate or other anionic surfactants should be improved significantly.

The object was achieved, surprisingly, by wash-active cosmetic and/or dermatological formulations comprising sodium PEG-7 olive oil carboxylate with an active content of 0.01 to 1.8% by weight, based on the total weight of the formulation, in addition to other auxiliaries, active compounds and additives.

### BRIEF DESCRIPTION OF THE DRAWINGS

FIG. 1 is graph illustrating sodium lauryl ether sulfate adsorption in the skin for various preparations including the preparations according to the invention.

### DETAILED DESCRIPTION OF THE PREFERRED EMBODIMENTS

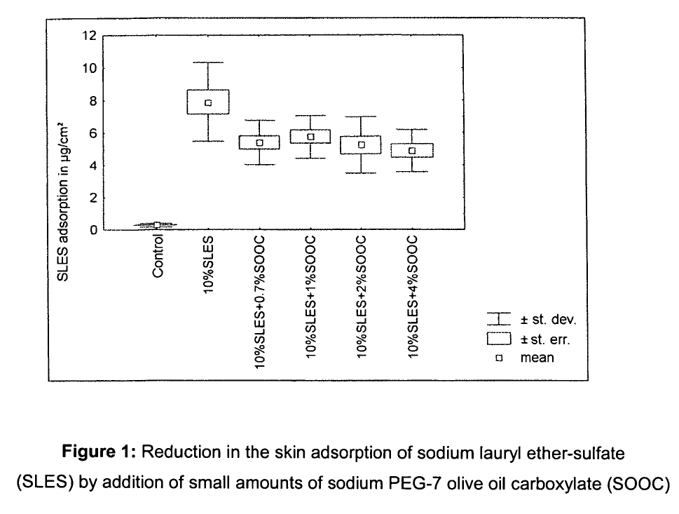

The use of amphoteric surfactants (e.g. cocamidopropylbetaine) to increase the skin tolerability of formulations comprising sodium laureth ether-sulfate is indeed known to the expert, but these formulations are distinguished by a high surfactant content in total, which is a disadvantage, inter alia, for economic and ecological reasons. In contrast, the formulations according to the invention render possible the formulation of skin-friendly formulations based on anionic surfactants with a reduced total content of surfactants. The formulations according to the invention are furthermore distinguished by a significantly improved foam quality. In particular, it was surprising that these effects are already to be achieved with the addition of less than 1.8% by weight, i.e. “catalytic”0 amounts, of sodium PEG-7 olive oil carboxylate (see FIG. 1).

The wash-active cosmetic and/or dermatological formulations comprise sodium PEG-7 olive oil carboxylate in a concentration of active content of 0.01 to 1.8% by weight, based on the total weight of the formulation.

The formulations according to the invention advantageously comprise sodium lauryl ether-sulfate in a concentration of 0.1 to 30% by weight, and particularly advantageously 7 to 12% by weight, in each case based on the total weight of the formulation.

The formulations according to the invention can furthermore advantageously comprise surfactants from the group consisting of N-acylamino acids and salts thereof in a concentration of 0.1 to 10% by weight, and particularly preferably 1 to 3% by weight, in each case based on the total weight of the formulation.

The formulations according to the invention are used for preventing or reducing the adsorption of sodium lauryl ether-sulfate on the human skin during the washing operation, for promoting desorption of sodium lauryl ether-sulfate from the human skin, for increasing the skin tolerability of wash-active cosmetic and/or dermatological formulations and for increasing the foam quality of cosmetic and/or dermatological formulations.

The formulations according to the invention are furthermore preferably used as a shower, foam and/or bath-tub bath and as a hair shampoo.

The compositions optionally comprise, according to the invention, in addition to the abovementioned surfactants, conventional additives in cosmetics, for example perfume, dyestuffs, antimicrobial substances, re-oiling agents, complexing and sequestering agents, pearlescent agents, plant extracts, vitamins, active compounds, preservatives, bactericides, pigments which have a coloring action, thickeners, softening, moisturizing and/or moisture-retaining substances, fats, oils, waxes or other conventional constituents of a cosmetic and/or dermatological formulation, such as alcohols, polyols, polymers, foam stabilizers, electrolytes, organic solvents or silicone derivatives.

The following examples are intended to illustrate the invention without limiting it. Unless stated otherwise, all the amounts data, contents and percentage contents are based on the weight and the total amount or on the total weight of the formulations.

### EXAMPLES

| | 1 | 2 | 3 | 4 | 5 | 6 |
|---|---|---|---|---|---|---|
| Sodium laureth | 9% | 9.5% | 9.5% | 9% | 11% | 10% |
| sulfate | | | | | | |
| PEG-7 olive | 1.5% | 1% | 1.8% | 1.7% | 0.5% | 1.8% |
| oil carboxylate | | | | | | |
| Cocoamido- | 3.65% | 3.9% | — | 2% | 3% | — |
| propylbetaine | | | | | | |
| Sodium | — | — | 2% | 1% | — | — |
| carboxymethyl- | | | | | | |
| cocoylpoly- | | | | | | |
| propylamine | | | | | | |
| Sodium cocoyl- | 1.8% | 2% | 1.75% | 1.7% | 2% | 1.5% |
| glutamate | | | | | | |
| PEG-40 | 0.50% | 0.50% | 0.50% | 0.50% | 0.50% | 0.50% |
| hydrogenated | | | | | | |
| castor oil | | | | | | |
| PEG-100 | 0.50% | 0.50% | 0.50% | 0.50% | 0.50% | 0.50% |
| hydrogenated | | | | | | |
| glyceryl | | | | | | |
| palmitate | | | | | | |
| Polyquater- | 0.05% | 0.1% | 0.2% | 0.2% | 0.1% | 0.15% |
| nium-10 | | | | | | |
| Sodium | 0.45% | 0.45% | 0.45% | 0.45% | 0.45% | 0.45% |
| benzoate | | | | | | |
| Sodium | 0.20% | 0.20% | 0.20% | 0.20% | 0.20% | 0.20% |
| salicylate | | | | | | |
| Citric acid | 0.50% | 0.50% | 0.50% | 0.50% | 0.50% | 0.50% |
| Perfume | q.s. | q.s. | q.s. | q.s. | q.s. | q.s. |
| Water | to 100 | to 100 | to 100 | to 100 | to 100 | to 100 |

That which is claimed:

1. A cosmetic or dermatological formulation, comprising sodium PEG-7 olive oil carboxylate.

2. The formulation as claimed in claim 1, wherein the sodium PEG-7 olive oil carboxylate is present in an active content of 0.01 to 1.8% by weight, based on the total weight of the formulation.

3. The formulation as claimed in claim 1, further comprising sodium lauryl ether sulfate in a concentration of 0.1 to 30% by weight, based on the total weight of the formulation.

4. The formulation as claimed in claim 1, further comprising sodium lauryl ether sulfate in a concentration of 7 to 12% by weight, based on the total weight of the formulation.

5. The formulation as claimed in claim 1, further comprising one or more surfactants from the group consisting of N-acylamino acids and salts thereof in a concentration of 0.1 to 10% by weight, based on the total weight of the formulation.

6. The formulation as claimed in claim 1, further comprising one or more surfactants from the group consisting of N-acylamino acids and salts thereof in a concentration of 1 to 3% by weight, based on the total weight of the formulation.

7. The formulation as claimed in claim 1, further comprising one or more additional compounds selected from the group consisting of auxiliaries, active compounds and additives.

8. A foam bath or shower product including the formulation as claimed in claim 1.

9. A hair shampoo including the formulation as claimed in claim 1.

10. A method for reducing the adsorption of sodium lauryl ether sulfate by the human skin, comprising applying to the skin a cosmetic or dermatological formulation comprising sodium PEG-7 olive oil carboxylate.

11. The method as claimed in claim 10, wherein the sodium PEG-7 olive oil carboxylate is present in an active content of 0.01 to 1.8% by weight, based on the total weight of the formulation.

12. The method as claimed in claim 11, wherein the formulation further comprises sodium lauryl ether sulfate in a concentration of 7 to 12% by weight, based on the total weight of the formulation.

13. The method as claimed in claim 10, wherein the formulation further comprises sodium lauryl ether sulfate.

14. A method for promoting desorption of sodium lauryl ether sulfate from the human skin, comprising applying to the skin a cosmetic or dermatological formulation comprising sodium PEG-7 olive oil carboxylate.

15. The method as claimed in claim 14, wherein the sodium PEG-7 olive oil carboxylate is present in an active content of 0.01 to 1.8% by weight, based on the total weight of the formulation.

16. A method for increasing the foam quality of cosmetic or dermatological formulations, comprising providing a cosmetic or dermatological formulation comprising sodium PEG-7 olive oil carboxylate.

17. The method as claimed in claim 16, wherein the sodium PEG-7 olive oil carboxylate is present in an active content of 0.01 to 1.8% by weight, based on the total weight of the formulation.

18. The method as claimed in claim 17, wherein the formulation further comprises sodium lauryl ether sulfate in a concentration of 7 to 12% by weight, based on the total weight of the formulation.

19. The method as claimed in claim 16, wherein the formulation further comprises sodium lauryl ether sulfate.

20. A method for increasing the skin tolerability of wash-active cosmetic or dermatological formulations, comprising providing a cosmetic or dermatological formulation comprising sodium PEG-7 olive oil carboxylate.

21. The method as claimed in claim 20, wherein the sodium PEG-7 olive oil carboxylate is present in an active content of 0.01 to 1.8% by weight, based on the total weight of the formulation.

22. The method as claimed in claim 21, wherein the formulation further comprises sodium lauryl ether sulfate in a concentration of 7 to 12% by weight, based on the total weight of the formulation.

23. The method as claimed in claim 20, wherein the formulation further comprises sodium lauryl ether sulfate.
